(19) United States
(12) Patent Application Publication
(10) Pub. No.: US 2016/0143709 A1
(43) Pub. Date: May 26, 2016

(54) IMPLANT COATING MATERIAL FOR ENHANCING A BIOACTIVITY AND OSSEOINTEGRATION OF IMPLANT SURFACE, AND THE METHOD FOR MANUFACTURING AND STORING THE SAME

(71) Applicant: OSSTEMIMPLANT CO., LTD., Seoul (KR)

(72) Inventors: II Seok Jang, Busan (KR); Su Kyoung KIM, Busan (KR); Ju Dong SONG, Busan (KR); Tae Gwan EOM, Busan (KR); Kyoo Ok CHOI, Seoul (KR)

(73) Assignee: OSSTEMIMPLANT CO., LTD., Seoul (KR)

(21) Appl. No.: 14/903,653

(22) PCT Filed: Oct. 25, 2013
(86) PCT No.: PCT/KR2013/009587

§ 371 Date: Jan. 8, 2016

(30) Foreign Application Priority Data
Jul. 18, 2013 (KR) 10-2013-0084899

(51) Int. Cl. A61C 8/00; A61L 27/54; A61L 27/06; A61L 27/28

(52) U.S. Cl. CPC A61C 8/0037; A61C 8/0013; A61L 27/28; A61L 27/54; A61L 27/06; A61C 2008/0046; A61L 2420/02; A61L 2300/224; A61L 2300/216; A61L 2400/18; A61L 2430/12

(57) ABSTRACT

A method for forming a coating film of a material for enhancing bioactivity, such as a water-soluble vitamin, on a surface of a dental implant, and a dental implant produced by the method, in order to overcome the limitations in improving osseointegration capability, which result from existing physical surface treatments, such as RBM and SLA surface treatments. The methods enable the SLA-pretreated implant surface to have blood affinity and an enhanced bioactive function, thereby providing a method for producing a dental implant which has excellent biocompatibility and high osseointegration capability after the implant procedure and thus has enhanced bioactivity, and a dental implant produced by the method. The surface of the dental implant, from which organic pollutants have been removed, are coated with a bioactive material, thereby enhancing blood affinity and bioactivity of the implant and thus improving initial osseointegration capability of the implant.

### TECHNICAL FIELD

The present invention relates to a method for preparing dental implant having an inhanced bioactivity and osseointegration properties, and provides a method for preparing the dental implant having the enhanced osseointegration properties by coating the bioactivity material to the surface of the implant to prevent a decrease of synosteosis performance due to a physical or chemical adsorption phenomenon of various contaminants such as hydrocarbon that a dental implant surface of titanium or titanium alloy material made by the existing conventional surface treatment method has, and to improve a bioactivity of a dental implant surface and a dental implant prepared by the same.

In addition, the present invention is to provide a method for preparing and storing a dental implant having the enhanced bioactivity and osseointegration properties to provide a method for stably storing it for a long time period and the dental implant stored by the same.

### BACKGROUND ART

As the surface treatment made of the implant widely used at present, Resorbable Blasted Media blasting(RBM) being a mechanical processing method, Sandblast Large grit Acid etch(SLA) being a chemical process method which treats the surface by an acidic material, etc. can be mentioned. Since the implant surface treated by such methods has a broad surface area and a high roughness over the surface treated by the existing common process method, there are advantages that it is advantageous for differentiation of osteoblast, and it can increase osseointegration between the bone and implant.

However, when the dental implant passed through the surface treatment process is exposed to the air, an oxidizing zone grows on the implant surface and the physical or chemical adsorption phenomenon of various contaminants such as hydrocarbon is occured, and thus the osseointegration performance may be reduced, and there is a limitation in further enhancing the osseointegration performance only by the surface treatment changing a micromorphology of implant surface (increse of surface area, inhancement of roughness).

In order to further enhance the physical osseointegration performance that titamium surface has, an approach coating the implant surface with osteoinduction protein, Bone morphogenetic protein (BMP) 2, osteogenesis peptide, fibrin gel, etc. has been made (Biomaterials 27 (2006) 5561-5571, Olin. Oral Implants Res. 2005 Oct; 16(5):563-9, Biomaterials 26 (2005) 5960-5971). But, although the material itself coated on the implant surface is an excellent material for inducing the differentiation of osteoblast and having the excellent bone induction performance, when really applying it on the surface of dental implant, there are problems such as no control of the release of (anything) on the coated implant surface or a reduction of efficiency when storing for a long time period due to the instability of a protein, the coating material, a denaturation due to gamma sterilization, etc.

Due to said problems, the applicant of the present invention has disclosed a method for treating and storing a surface of dental titanium implant, which is characterized in that the surface of titanium implant is treated with UV-ozon to modify it into superhydrophilic surface, and then immersing the implant into a solution comprising the specific kinds of divalent salt selected for maintaining the hydrophilicity of the modified surface (Korean Patent No. 10-1271402).

### DISCLOSURE

### Technical Problem

The present invention is to overcome the limitation of the enhancement of osseointegration performance that the method for treating physical surface such as the existing RBM and SLR surface has, and provide the method for forming the coating membrane of the material enhancing the bioactivity such as water soluble vitamin into the dental implant surface and the dental implant Prepared by the same.

Through the method, since a blood-affinity can be imparted to the SLA pre-treated implant surface and physiological activity function can be improved, the method preparing the dental implant having the improved physiological activity and the dental implant prepared by the same are provided. In addition, the method for storing the dental implant so as to enhance the bioactivity and osseointegration properties and the dental implant stored by the same are provided.

### Technical Solution

The present invention relates to the method for preparing the dental implant having the enhanced bioactivity and osseointegration properties, and a method for preparing the dental implant having the enhanced bioactivity and osseointegration properties, which is characterized by comprising step 1 for forming the roughed surface on the dental implant surface of titanium or titanium alloy material; step 2 for removing the contaminants on the said roughed dental implant surface; step 3 for forming a coating membrane of the material enhancing the bioactivity on the above roughed dental implant surface; and step 4 for drying the above dental implant surface on which the coating membrane of the material enhancing the bioactivity are formed.

In the above step for forming the coating membrane of the material enhancing the bioactivity, the aqueous solution of the material enhancing the bioactivity, which is coated on the implant surface, has a concentration of 0.01˜1 M, and the aqueous solution can be used in the range of pH 4˜8.

As a material enhancing the bioactivity of the dental implant surface in the present invention, water soluble vitamin B, water soluble vitamin C or the derivatives thereof can be used, and preferably at least any one or more of water soluble vitamin B1, water soluble vitamin B6, water soluble vitamin B12 or water soluble vitamin C can be used.

As other embodiment of the present invention, there is a method for preparing and storing the dental implant having the enhanced bioactivity and osseointegration properties, which comprises step 1 for forming the roughen surface on the dental implant surface; step 2 for removing contaminants on said roughen dental implant surface; and step 3 for immersing and storing said roughen dental implant in the aqueous solution of the material enhancing the bioactivity.

As other embodiment of the present invention, there are the implant prepared by the method for preparing the dental implant having the enhanced bioactivity and osseointegration properties, and the dental implant which is stored by the method storing the dental implant to enhance the bioactivity and osseointegration properties

### Advantageous Effects

According to the metal implant and its preparation of the present invention, the dental implant which the organic contaminants of the surface is removed, has the enhanced blood-affinity and bioactivity by coating its surface with materials enhancing the bioactivity to improve the initial osseointegration performance.

Also, since the implant is prepared by coating and drying the water soluble vitamins, when storing it for a long time period, a stable coating layer is formed and the blood-affinity and bioactivity on the implant surface are exhibited, and thus, it has effects that the initial osseointegration performance is improved after the implant surgery and eventually the time period for bone-integrating is shortened.

### DESCRIPTION OF DRAWINGS

FIG. 1 is a SEM photograph for the dental implant surface after coating it with the materials enhancing the bioactivity of the present invention and drying it, wherein SA represents the uncoated implant, and Vit B1, Vit B6, Vit B12 and Vit C represent the implants coated by Vitamin B1, Vitamin B6, Vitamin B12, Vitamin C, respectively.

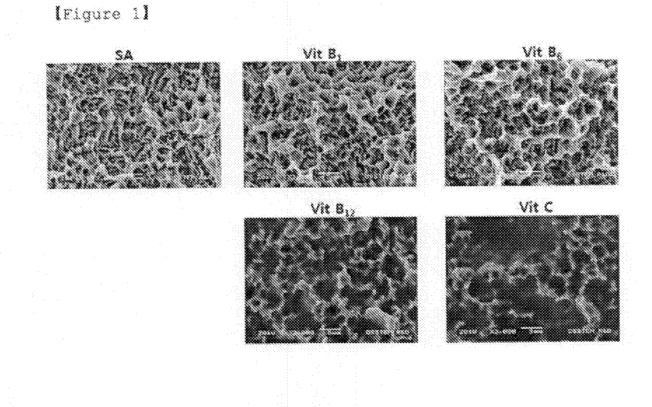

FIG. 2 is an experimental result determining the bond-interface binding force of the dental implant with acceleration aging of 1 year.

FIG. 3 is an experimental result determining the bond-interface binding force of the dental implant with acceleration aging of 3 year.

FIG. 4 is an experimental result determining the blood-affinity for dental implant coated with water soluble Vitamin (B group, C).

FIG. 5 is a SEM photograph identifying the micromorphology formed after acid treatment of a machined titanium cylinder.

FIG. 6 is a metrological assessment result for the tissue of the bond interface of titanium cylinder coated with materials enhancing the bioactivity.

### BEST MODE FOR INVENTION

The present invention is to inhibit the adsorption of the contaminants which cause the surface to be hydrophobic, and thus to keep the super-hydrophilic property of the dental implant surface, by forming coating membrane of materials enhancing the bioactivity such as water soluble Vitamin B, water soluble Vitamin C or the derivatives thereof on the dental implant surface on which the contaminants are removed, as well as, to have an excellent osseointegration performance after the implant surgery, by enhancing the bioactivity of the dental implant surface.

In order to achieve them, the method of the present invention comprises step 1 for forming the roughed surface on the dental implant surface made of titanium or titanium alloy material; step 2 for removing the contaminants on said roughed dental implant surface; step 3 for forming the coating membrane of materials enhancing the bioactivity on said roughed dental implant surface; and step 4 for drying said dental implant surface on which the coating membrane of materials enhancing the bioactivity is formed.

In addition, in order to obtain the effect for storing it for a long time period, with making the method for forming on the dental implant surface the coating membrane of materials enhancing the bioactivity such as water soluble Vitamin B, water soluble Vitamin C or the derivatives thereof more easy, it is preferable to store the dental implant prepared so as for the coating step to be naturally made in the storage solution for the storage Period of the implant, by immersing the dental implant passed through the step for removing contaminants in the container with the aqueous solution of materials enhancing the bioactivity such as said water soluble Vitamin B, water soluble Vitamin C or the derivatives thereof after the roughening treatment of the surface and by closing the container.

In the method for preparing the dental implant of the present invention, it is preferable to use the aqueous solution of materials enhancing the bioactivity such as water soluble Vitamin B, water soluble Vitamin C or the derivatives thereof so as for the outer surface of the dental implant to be uniformly coated.

Hereinafter, the present invention will be explained more in detail through the Working Examples.

### EXAMPLE 1

The machined dental implant was blasted with Al₂O₃ powder having the particle size of 1 mm or less at the blast pressure of 1˜10 atmosphere for 1˜60 seconds. The macro and micro morphology via the acid treatment using the mixed acid aqueous solution was imparted to the implant surface. After washing said acid-etched dental implant with ethanol for 30 minutes, it was performed by ultrasonic washing with distilled water for 30 minutes and dried.

The contaminants which adsorbed or stabilized on the dental implant surface were removed by treating the dental implant passed through said steps for 1 minute with plasma or for 5 minutes with ultraviolet ray, and the dental implant surface was uniformly coated with 5 μl of concentration of 0.01˜1M water soluble Vitamin B group or water soluble Vitamin C aqueous solution and then was dried at the room temperature.

The implant on which water soluble Vitamin (B group or C) is coated, as prepared by such method, was used in Example 2 below.

### EXAMPLE 2

After leaving the dental implant wherein water soluble Vitamin (B group and C) was coated and then dried, as prepared in said Example 1, under the acceleration aging condition (about 55° C.) for six weeks (1 year of the acceleration aging), in order to determine the implant-bond interface binding force, the implant was inserted to micropig tibia and then a removal torque was determined. At this time, the implant wherein said materials were not coated was used as a control.

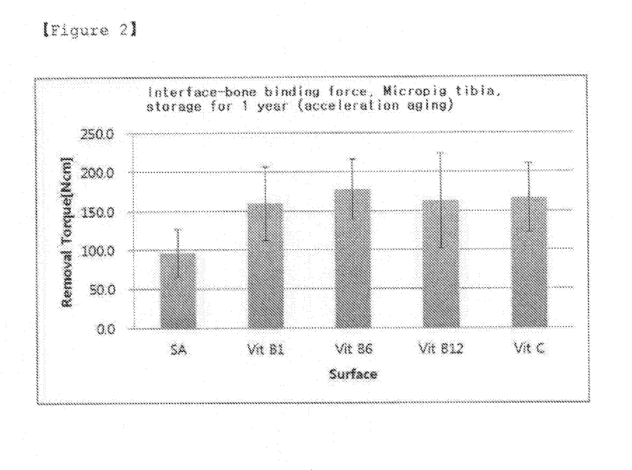

As shown in FIG. 2, it can be noticed that in the case of the acceleration aging of 6 weeks (1 year of acceleration aging), the experimental group wherein water soluble Vitamin (B group and C), Vitamin B1, Vitamin B6, Vitamin B12, and Vitamin C were coated and dried, exhibited the enhanced bone-interface binding force, with the removal torque being the extent of about 66%, 85%, 69% and 73%, respectively, over the control.

### EXAMPLE 3

After leaving the dental implant wherein water soluble Vitamin (B group and C) was coated and then dried, as prepared in said Example 1, under the acceleration aging condition (about 55° C.) for eighteen weeks (3 years of the acceleration aging), in order to determine the implant-bond interface binding force, the implant was inserted to micropig tibia and then the removal torque was determined after osteogenesis period of 16 days. In this case, the implant wherein said materials were not coated was used as a control.

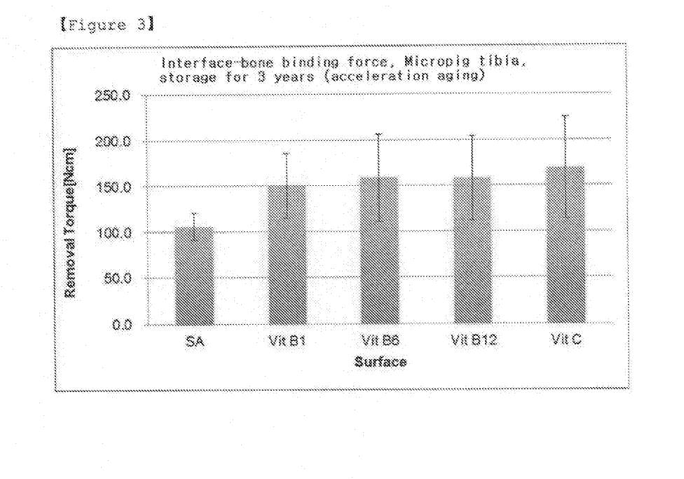

As shown in FIG. 3, it can be noticed that although the acceleration aging of 18 weeks (3 years of acceleration aging) was performed, the experimental group wherein water soluble Vitamin (B group and C), Vitamin B1, Vitamin B6, Vitamin B12, and Vitamin C were coated and dried, exhibited the enhanced bone surface coherence as in the 6 weeks of accelerated aging, with the removal torque being the extent of about 42%, 50%, 49% and 60%, respectively, over the control.

### EXAMPLE 4

After leaving the dental implant prepared in said Example 3 under the acceleration aging condition (about 55° C.) for eighteen weeks, in order to identify the blood-affinity, the implant was immersed in a depth of about 3 mm in micropig blood for the predetermined time (30 seconds), and then, a wetting property of blood was identified as the height of the blood coming up along the implant surface. In this case, the implant wherein pre-treatment process imposing hydrophilic property was not made, and said materials were not coated, was used as a control.

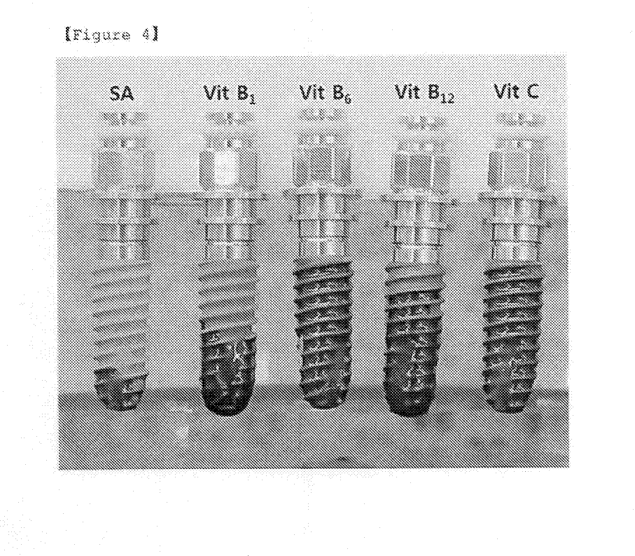

As shown in FIG. 4, it can be visually noticed that the implant dried after coating with water soluble Vitamin, Vitamin B1, Vitamin B6, Vitamin B12 and Vitamin C, exhibited the higher wetting of blood than the control, and such surface treatment process has the blood-affinity. Such blood-affinity was increased just after the preparation of it, but especially the margin of increase was maintained in the experiment group over the control group even after the lapse of 3 years.

### EXAMPLE 5

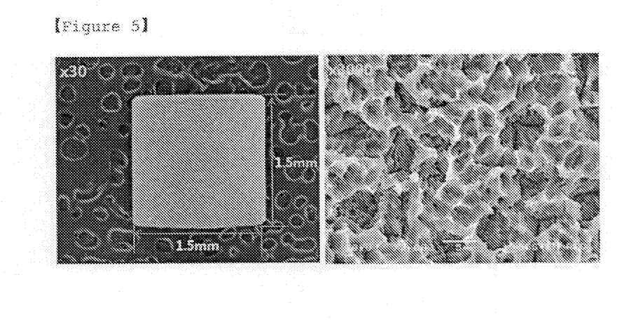

A micro morphology was imparted to the surface of the machined titanium cylinder having 1.5 mm diameter×1.5 mm length by applying it to the acid treatment with the mixed acid aqueous solution (Refer to FIG. 5). After washing the titanium cylinder treated by acid-etching with ethanol for 30 minutes, it was applied to an ultrasonic washing with distilled water and then was dried.

The titanium cylinder passing through the said process was treated with plasma 1 minute or ultraviolet ray for 5 minutes to impart the hydrophilic property, with removing the contaminants adsorbed on the surface, the surface was uniformly coated with 1 ml of aqueous solution of 0.01˜1 M Vitamin (B group, C), Vitamin B1, Vitamin B6, Vitamin B12, Vitamin C and then was dried.

The titanium cylinder wherein the water soluble Vitamin (B group, C), Vitamin B1, Vitamin B6, Vitamin B12, Vitamin C was coating-dried, as prepared by such method, was used in Example 6 below.

### EXAMPLE 6

After leaving the dental implant prepared in said Example 5 under the acceleration aging condition (about 55° C.) for eighteen weeks, in order for the tissue metrological evaluation of the bone surface, the titanium cylinder was inserted to rat tibia, osteogenesis period of 14 days was passed and then, tissue slides were made and the tissue metrological evaluation was performed. At this time, titanium cylinder wherein said materials were not coated was used as a control.

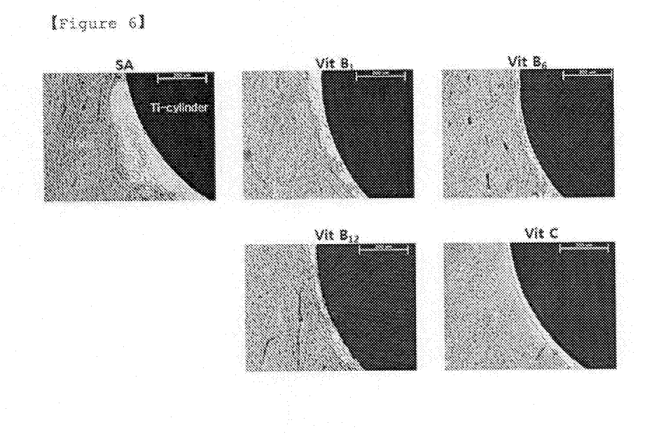

As shown in FIG. 6, despite the acceleration aging of weeks, it can be identified that in the titanium cylinder wherein water soluble Vitamin (Vitamin B group, C), Vitamin B1, Vitamin B6, Vitamin B12 and Vitamin C were coated and dried, Bone to Implant Contact (BIC) and Bone Area (BA) in the periphery part of titanium cylinder were greatly increased over the control.

### INDUSTRIAL APPLICABILITY

The present invention is to overcome the limitation of osseointegration performance enhancement in the physical surface treatment method such as the existing RBM and SLA surfaces, and is to provide the method for forming the coating membrane of materials enhancing the bioactivity such as water soluble Vitamin and the dental implant prepared by such method. Since a blood-affinity can be imparted to the SLA pre-treated implant surface and the bioactivity function can be improved through such method, there is provided the method for preparing the dental implant enhancing bioactivity, which has an excellent biocompatibility after the implant surgery and the high osseointegration performance period, and the dental implant prepared by such method, and the dental implant wherein the organic contaminants on the implant surface are removed, is increased in the blood-affinity and physiological activity by coating its surface with physiological active materials and can enhance the initial osseointegration, and thus, it has the Industrial applicability.

1. A method for preparing a dental implant having an enhanced bioactivity and osseointegration properties, the method comprising:
step 1 for forming a roughed surface on the dental implant surface made of titanium or titanium alloy;
step 2 for removing contaminants on the roughed dental implant surface;
step 3 for forming on the roughed dental implant surface wherein contaminants are removed, a coating membrane with a aqueous solution of materials enhancing the bioactivity, which comprises the materials enhancing the bioactivity selected from water soluble Vitamin B, water soluble Vitamin C and the derivatives thereof in the concentration of 0.01˜1 M, and has pH in the range of 4˜8; and
step 4 for drying the dental implant surface on which the coating membrane of the materials enhancing the bioactivity is formed.

2. The method of claim 1, wherein the materials enhancing the bioactivity is one or more of water soluble Vitamin B1, water soluble Vitamin B6, water soluble Vitamin B12 or water soluble Vitamin C.

3. A method for storing the dental implant having the enhanced bioactivity and osseointegration properties, the method comprising:
step 1 for forming the roughed surface on the dental implant surface made of titanium or titanium alloy;
step 2 for removing the contaminants on the said roughed dental implant surface; and
step 3 for storing the roughed dental implant wherein the contaminants are removed by immersing it in the aqueous solution of the material enhancing the bioactivity which comprises material enhancing the bioactivity selected from water soluble Vitamin B, water soluble Vitamin C and the derivatives thereof in a concentration of 0.01˜1 M and has pH in the range of 4˜8, in the method for preparing the dental implant having the enhanced bioactivity and osseointegration properties and for storing it.

4. The method of claim 3, wherein the material enhancing the bioactivity is one or more of water soluble Vitamin B1, water soluble vitamin B6, water soluble Vitamin B12 or water soluble Vitamin C.

5. The method of claim 1, wherein the step 1 for forming the roughed surface comprises blasting the implant with Al₂O₃ powder having the particle size of 1 mm or less at the blast pressure of 1˜10 atmosphere for 1˜60 seconds.

6. The method of claim 3, wherein the step 1 for forming the roughed surface comprises blasting the implant with Al₂O₃ powder having the particle size of 1 mm or less at the blast pressure of 1˜10 atmosphere for 1˜60 seconds.

7. The method of claim 1, wherein the step 2 for removing contaminants comprises removing the contaminants adsorbed on the implant surface by treating the dental implant with plasma for 1 minute or with ultraviolet ray for 5 minutes.

8. The method of claim 3, wherein the step 2 for removing contaminants comprises removing the contaminants adsorbed on the implant surface by treating the dental implant with plasma for 1 minute or with ultraviolet ray for 5 minutes.

9. A dental implant prepared by the method of claim 1.

10. A dental implant prepared by the method of claim 3.
